(19) United States
(12) Patent Application Publication
(10) Pub. No.: US 2008/0077184 A1
(43) Pub. Date: Mar. 27, 2008

(54) Intravascular Stimulation System With Wireless Power Supply

(76) Inventors: Stephen Denker, Mequon, WI (US); Cherik Bulkes, Sussex, WI (US)

Correspondence Address: QUARLES & BRADY LLP, 411 E. WISCONSIN AVENUE, SUITE 2040, MILWAUKEE, WI, 53202-4497, US

(21) Appl. No.: 11/535,504

(22) Filed: Sep. 27, 2006

(51) Int. Cl. A61N 1/18; A61N 1/00

(52) U.S. Cl. 607/2

(57) ABSTRACT

A medical device adapted for implantation into a patient receives electrical power from an extravascular power supply. The medical device has a first receiver for a first radio frequency (RF) signal from which energy is extracted to power the medical device, and a second RF signal carries an indication of an amount of that extracted energy. The extravascular power supply includes a source of electrical power and a power transmitter that emits the first RF signal which is varied in response to the indication from the second radio frequency signal. Animal physiological data also can be carried by the second RF signal. The medical device includes a system that monitors the effects of tissue stimulation and regulates subsequent stimulation accordingly.

### FIELD OF THE INVENTION

The present invention relates to implantable medical devices which deliver energy to stimulate tissue in an animal, and more particularly to transvascular simulation in which the medical device is implanted in a vein or artery to stimulate the adjacent tissue or organ.

### DESCRIPTION OF THE RELATED ART

A remedy for people with slowed or disrupted natural heart activity is to implant a cardiac pacing device which is a small electronic apparatus that stimulates the heart to beat at regular rates.

Typically the pacing device is implanted in the patient's chest and has sensor electodes that detect electrical impulses associated with in the heart contractions. These sensed impulses are analyzed to determine when abnormal cardiac activity occurs, in which event a pulse generator is triggered to produce electrical pulses. Wires carry these pulses to electrodes placed adjacent specific cardiac muscles, which when electrically stimulated contract the heart chambers. It is important that the stimulation electrodes be properly located to produce contraction of the heart chambers.

Modern cardiac pacing devices vary the stimulation to adapt the heart rate to the patient's level of activity, thereby mimicking the heart's natural activity. The pulse generator modifies that rate by tracking the activity of the sinus node of the heart or by responding to other sensor signals that indicate body motion or respiration rate.

U.S. Pat. No. 6,445,953 describes a cardiac pacemaker that has a pacing device, which can be located outside the patient, to detect abnormal electrical cardiac activity. In that event, the pacing device emits a radio frequency signal that is received by a circuit mounted on a stimulator body implanted in a vein or artery of the patient's heart. Specifically, the radio frequency signal induces a voltage pulse in an antenna and that pulse is applied across a pair of electrodes on the body, thereby stimulating adjacent muscles and contracting the heart. Although this cardiac pacing apparatus offered several advantages over other types of pacemakers, it required placement of sensing electrodes on the patient's chest in order for the external pacing device to detect when the heart requires stimulation.

### SUMMARY OF THE INVENTION

An apparatus is provided for artificially stimulating internal tissue of an animal by means of an intravascular medical device adapted for implantation into the animal's blood vasculature. The intravascular medical device comprises a power supply and first and second stimulation electrodes for contacting the tissue. A control circuit governs operation of a stimulation signal generator connected to the first and second stimulation electrodes. The stimulation signal generator produces a series of electrical stimulation pulses and a voltage intensifier increases the voltage of each electrical stimulation pulse to produce an output pulse that is applied to the first and second stimulation electrodes.

The voltage intensifier can use any of several techniques to increase the stimulation pulse voltage. Preferably, flying capacitor type voltage doubling, bipolar mode doubling, or a combination of both is used.

One version of the medical device includes a mechanism that is connected to the first and second stimulation electrodes for sensing effects from the electrical stimulation pulse and producing a feedback signal indicating such effects. The stimulation pulses are altered in response to the feedback signal, thereby controlling stimulation of the tissue.

The apparatus includes an extravascular power source that transmits a first wireless signal conveying electrical energy to power the medical device. Circuitry in the medical device extracts energy from the first wireless signal for use by the power supply. A feedback transmitter in the medical device transmits a second wireless signal carrying an indication of the amount of that extracted energy. The extravascular power source receives the second wireless signal and uses the indication as a feedback signal to control the amount of energy conveyed by the first wireless signal. In the preferred embodiment, the first wireless signal is pulse width modulated to vary the amount of conveyed energy.

The first wireless signal has another type of modulation that encodes operating commands which are sent from the extravascular power supply to the intravascular medical device. The intravascular medical device also can sense a physiological characteristic of the animal and send data related to the physiological characteristic via the second wireless signal. In that case, the second wireless signal has a first type of modulation that carries the indication of an amount of the extracted energy and a second type of modulation that carries the physiological characteristic data.

### BRIEF DESCRIPTION OF THE DRAWINGS

FIG. 1 is a representation of a cardiac pacing system that includes an extravascular power supply and an intravascular medical device attached to a medical patient;

FIG. 2 is an isometric, cut-away view of a patient's blood vessels in which a receiver antenna, a stimulator and an electrode of the intravascular medical device have been implanted at different locations;

FIG. 3 is a block schematic diagram of the electrical circuitry for the cardiac pacing system;

FIG. 4 is a schematic diagram of a voltage intensifier in the intravascular medical device; and

FIG. 5 is a schematic diagram of a voltage inverter;

FIG. 6 illustrates the waveform of a radio frequency signal by which energy and data are transmitted to the intravascular medical device;

FIGS. 7A and B are waveform diagrams of the power supply signal and data respectively recovered from a radio frequency signal received by the intravascular medical device;

FIGS. 8A and B are pulse trains transmitted from the intravascular medical device to an external receiver containing information pertaining to the level of the power supply signal and to sensed physiological data for the medical patient; and

FIG. 9 depicts waveform diagrams related to bipolar stimulation signal generation.

### DETAILED DESCRIPTION OF THE INVENTION

Although the present invention is being described in the context of cardiac pacing and of implanting a stimulator in a vein or artery of the heart, the present apparatus can be employed to stimulate other areas of the human body. In addition to cardiac applications, the stimulation apparatus can provide brain stimulation, for treatment of Parkinson's disease or obsessive/compulsive disorder for example. The transvascular electrical stimulation also may be applied to muscles, the spine, the gastro/intestinal tract, the pancreas, and the sacral nerve. The apparatus may also be used for GERD treatment, endotracheal stimulation, pelvic floor stimulation, treatment of obstructive airway disorder and apnea, molecular therapy delivery stimulation, chronic constipation treatment, and electrical stimulation for bone healing.

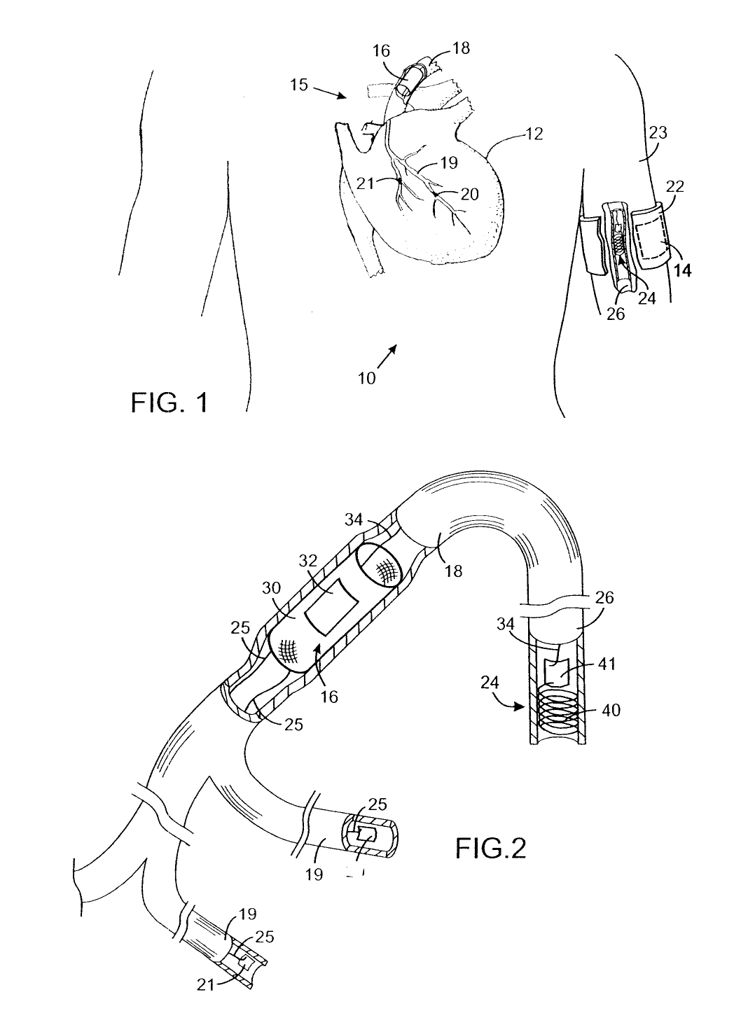

Initially referring to FIG. 1, a medical apparatus, in the form of a cardiac pacing system 10 for electrically stimulating a heart 12 to contract, comprises a power transmitter 14, preferably worn outside the patient's body, and a medical device 15 implanted in the circulatory system of a human patient 11. Alternatively the power transmitter 14 may be implanted in the patient. The medical device 15 receives a radio frequency (RF) signal from the extracorporeal power transmitter 14 and the implanted electrical circuitry is electrically powered by the energy of that signal. Thus the power transmitter 14 acts as a power source for the implanted medical device 15. At appropriate times, the medical device 15 delivers an electrical stimulation pulse into the surrounding tissue of the patient thereby producing a contraction of the heart 12.

Referring to FIGS. 1 and 2, the exemplary implanted medical device 15 includes an intravascular stimulator 16 located in a vein or artery 18 in close proximity to the heart 12. One or more electrical wires 25 lead from the stimulator 16 through the cardiac blood vasculature to locations in smaller blood vessels 19 at which stimulation of the heart is desired. At such locations, the electrical wire 25 is connected to a remote electrode 21 secured to the blood vessel wall so as to have better transfer efficiency than when if the electrode floats in the blood pool. The electrodes 5 may be placed proximate to the sinus node (e.g. in the coronary sinus vein), the atria, or the ventricles of the heart, for example.

Because the stimulator 16 of the medical device 15 is near the heart and relatively deep in the chest of the human medical patient, an assembly 24 of transmit and receive antennas for radio frequency signals are preferably implanted in a vein or artery 26 of the patient's upper right arm 23. The antenna assembly 24 is connected to the stimulator 16 by a cable 34. The arm vein or artery 26 is significantly closer to the skin and thus antenna assembly 24 picks up a greater amount of the energy of the radio frequency signal emitted by the extracorporeal power transmitter 14, than if the antenna assembly was located on the stimulator 16. Preferably, the power transmitter 14 is mounted on a single flexible circuit board in a patch or arm band 22 on the patient's arm in close proximity to the location of the antenna assembly 24. Alternatively, another limb, neck or other area of the body with an adequately sized blood vessel close to the skin surface of the patient can be used. Alternatively, an extravascular power transmitter 14 may be implanted in the patient outside the blood vessels. As used herein, the adjective “extravascular” includes extracorporeal items unless further qualified.

As illustrated in FIG. 2, the intravascular stimulator 16 has a body 30 constructed similar to well-known expandable vascular stents. The stimulator body 30 comprises a plurality of wires formed to have a memory defining a tubular shape or envelope. Those wires may be heat-treated platinum, Nitinol, a Nitinol alloy wire, stainless steel, plastic wires or other materials. Plastic or substantially nonmetallic wires may be loaded with a radiopaque substance which provides visibility with conventional fluoroscopy. The stimulator body 30 has a memory so that it normally assumes an expanded configuration when unconfined, but is capable of assuming a collapsed configuration when disposed and confined within a catheter assembly, as will be described. In that collapsed state, the tubular body 30 has a relatively small diameter enabling it to pass freely through the blood vasculature of a patient. After being properly positioned in the desired blood vessel, the body 30 is released from the catheter and expands to engage the blood vessel wall. The stimulator body 30 and other components of the medical device 15 are implanted in the patient's circulatory system a catheter.

The body 30 has a stimulation circuit 32 mounted thereon and connected to first and second stimulation electrodes 20 and 21 located remotely in small cardiac blood vessels. The stimulation electrodes 20 and 21 can be embedded directly in the blood vessel wall or mounted on a collapsible body of the same type as the stimulator body 30. It should be understood that additional stimulation electrodes can be provided with the stimulation circuit selectively applying electrical pulses across different pairs of those electrodes to stimulate respective regions of the patient's tissue.

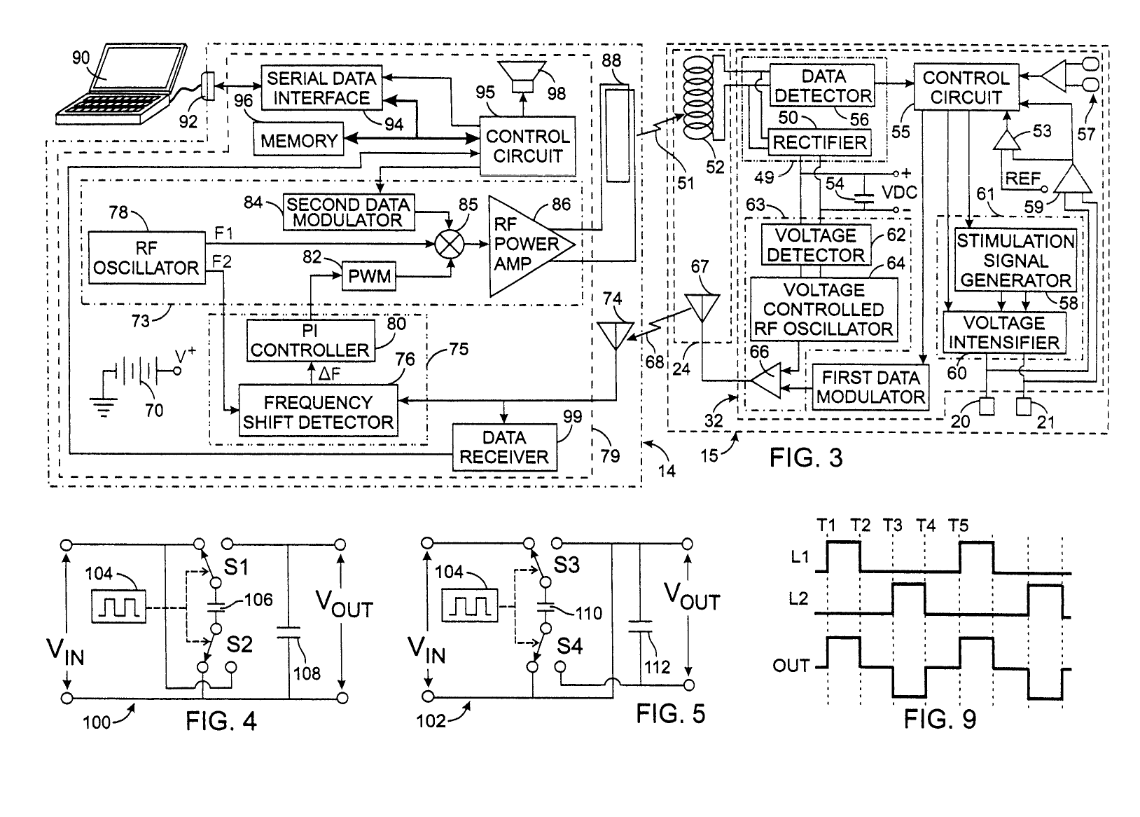

With reference to FIG. 3, the stimulation circuit 32 includes a first receive antenna 52 within the antenna assembly 24 and that antenna is tuned to pick-up a first wireless signal 51. The first wireless signal 51 provides electrical power and carries control commands to the medical device 15. FIG. 6 depicts the format of the wireless signal 51. The first wireless signal 51 comprises a periodically occurring power pulse 46 of a signal at a first radio frequency (F1) that preferably is less than 50 MHz to prevent excessive RF losses in the tissue of the patient. The power pulses 46 are pulse width modulated to control the amount of power applied to the medical device 15. The pulse width modulation is manipulated to control the amount of energy the medical device receives to ensure that it is sufficiently powered without wasting energy from the battery 70 in the power transmitter 14. Alternatively the frequency of the pulses within the burst can be frequency modulated to similarly control the amount of power.

The first receive antenna 52 is coupled to a discriminator 49 that separates the signal received by the antenna into RF power and data. A rectifier 50 in the discriminator 49 functions as a power circuit that extracts energy from the received first wireless signal. Specifically, the radio frequency, first wireless signal 51 is rectified to produce a DC voltage (VDC) that is applied across a storage capacitor 54 which functions as a power supply by furnishing electrical power to the other components of the medical device.

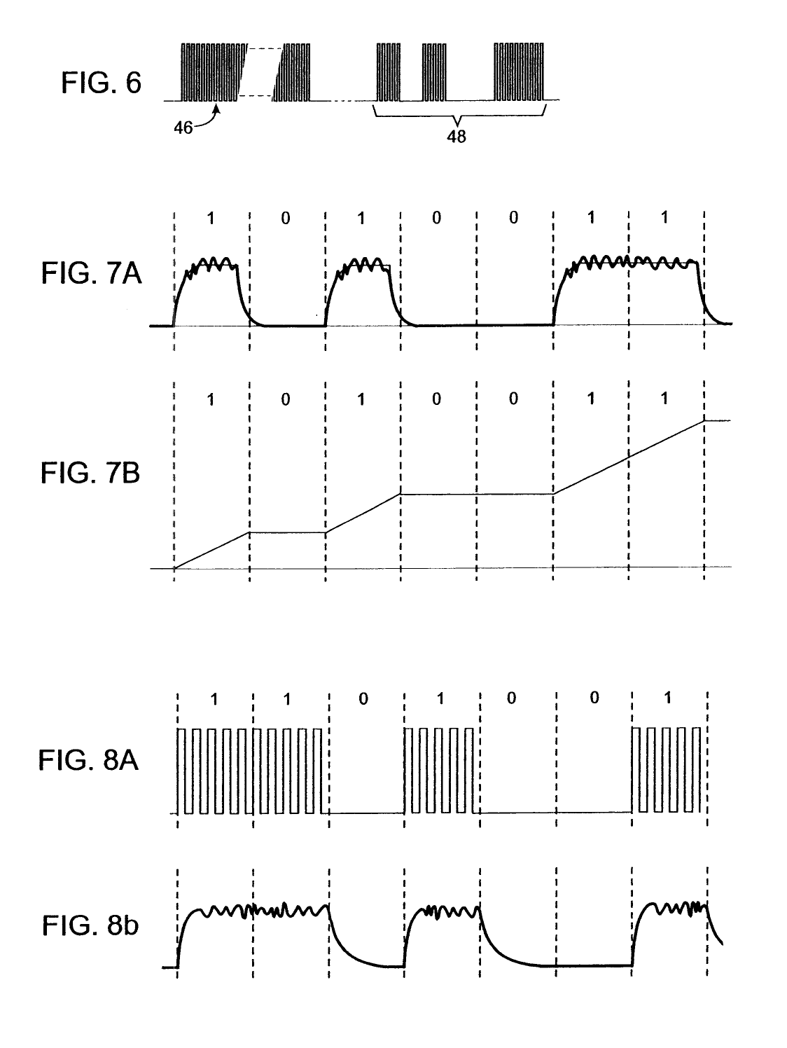

As necessary the first wireless signal 51 also carries control commands that specify operational parameters of the medical device 15, such as the duration of a stimulation pulse that is applied to the electrodes 20 and 21. Those commands are sent digitally as a series of binary bits encoded on the first wireless signal 51 by fixed duration pulses 48 of the first radio frequency signal. The amplitude of the envelopes varies to modulate the control command bits on the first radio frequency signal. The first receive antenna 52 is coupled to a discriminator 49 that separates the signal received by the antenna into RF power and data. That discriminator 49 includes a data detector 56 that recovers data and commands carried by the first wireless signal 51. FIG. 7A illustrates the data pulse train as it appears after recovery by the data detector 56. That detector incorporates a rectifier/capacitor circuit which suppresses the RF carrier except for the small ripple shown, however the capacitor is relatively small to have minimal affect on the data pulses except for the time constant effect on the leading and trailing edges.

The recovered data is sent to a control circuit 55 for that medical device, which stores the operational parameters for use in controlling operation of a stimulator 61 that applies tissue stimulating voltages pulses across the electrodes 20 and 21. Preferably, the control circuit 55 comprises a conventional microcomputer that has analog and digital input/output circuits and an internal memory that stores a software control program and data gathered and used by that program.

The control circuit 55 also receives data from a pair of sensor electrodes 57 that detect electrical activity of the heart and provide conventional electrocardiogram signals which are utilized to determine when cardiac pacing should occur. Additional sensors for other physiological characteristics, such as temperature, blood pressure or blood flow, may be provided and connected to the control circuit 55. The control circuit stores a histogram of pacing data related to usage of the medical device and other information which can be communicated to the power transmitter 14 or another form of a data gathering device that is external to the patient 11, as will be described.

### Stimulation Signal Regulation

The software executed by the control circuit analyzes the electrocardiogram signals and other physiological characteristics from the sensor electrodes 57 to determine when to stimulate the patient's heart. As noted previously the present system can be used to stimulate other regions of the patient's body, such as the brain for treatment of Parkinson's disease or obsessive/compulsive disorder, muscles, the spine, the gastro/intestinal tract, the pancreas, and the sacral nerve, to name a few examples, in which case the sensor electrodes 57 detect physiological characteristics associated with those regions. When stimulation is required the control circuit 55 issues a command to the stimulator 61 which comprises a stimulation signal generator 58 that responds by applying one or more pulses of voltage from the storage capacitor 54 across various pairs of the electrodes 20 and 21 depending upon which area of the heart 12 is to be stimulated. The stimulation signal generator 58 controls the intensity and shape of the pulses. The output pulses from the stimulation signal generator 58 can be applied either directly to those electrodes 20 and 21 or via an optional voltage intensifier 60.

The voltage intensifier 60 preferably is a “flying capacitor” inverter that charges and discharges in a manner that essentially doubles the power. This type of device has been used in integrated circuits for local generation of additional voltage levels from a single supply. FIGS. 6A and 6B respectively illustrated the doubler and inverter stages 100 and 102 of the voltage intensifier 60. In the doubler stage 100 of FIG. 6A, a pair of switches S1 and S2 are operated by a square wave signal from a generator 104 to alternately charge and discharge an input capacitor 106 with the input voltage VIN. When the switches S1 and S2 are positioned as shown, the input capacitor 106 is charge by the input voltage VIN. During the discharge part of the switch cycle, the voltage across the input capacitor 106 added to the voltage already across an output capacitor 108, that is connected between the output terminals of the doubler stage 100. In the inverter stage 102 of FIG. 6B, a second pair of switches S3 and S4 are operated by the square wave signal from the generator 104 to alternately charge and discharge an input capacitor 106 with the input voltage VIN to the inverter. During the discharge part of the switch cycle of this circuit, the voltage on the input capacitor 110 is applied across the output capacitor 112 and the output terminals in a manner that inverts the polarity of the output voltage VOUT with respect to the input voltage VIN. A doubler stage 100 and an inverter stage 102 can be connected in series to produce an increased inverted output voltage to apply to a pair of the stimulation electrodes 20 and 21. When there are more that two stimulation electrodes a switching circuit is provided at the output of the voltage intensifier 60 to selectively apply the output voltage VOUT across one pair of those electrodes. Various numbers of doubler stages 100 can be concatenated to increase the voltage from the storage capacitor 54 to the desired stimulation output voltage. The number of doubler stages may be switchable in response to control signals from the control circuit 55 thereby enabling the voltage to be increase by different powers of two and inverted without use of inductors. The voltage intensifier 60 also has switches operated by the control circuit 55 to connect the stimulation output voltage to a selected pair of the electrodes in order to stimulate a particular region of the heart.

The stimulation voltages also can be doubled by bipolar mode operation since the circuit is not externally grounded. This is accomplished without using transformers, inverters or converters. For unipolar operation one output line L1 is always connected to the negative terminal of the storage capacitor 54 and another output line L2 is switched between the negative and positive terminals of the storage capacitor 54. This varies the voltage between those output lines and thus between a pair of stimulation electrodes from 0 to VDC where output line L2 is either the same voltage as or positive with respect to L1.

FIG. 9 depicts bipolar operation in which both output lines L1 and L2 are switched between the negative and positive terminals of the storage capacitor 54. In other words each output line is switched between 0 and VDC. However, both output lines are never connected simultaneously to the positive terminal. Initially both output lines L1 and L2 are connected to the negative terminal which is arbitrarily defined as the zero volt level. Alternatively, the positive terminal could be defined as the zero volt level in which case both output lines are never connected to the negative terminal simultaneously. At time T1, the output line L1 is switched to the positive terminal while output line L2 remains connected to the negative terminal, thereby rendering L1 positive with respect to L2 by VDC. Then at time T2, output line L1 is switched to the negative terminal and output line L2 which returns both lines to zero. Next output line L2 is switched to the positive terminal at time T3 while output line L1 remains connected to the negative terminal, thereby rendering L2 positive with respect to L1 by VDC. At time T4 both output lines are connected to the negative terminal. The switching pattern repeats successively beginning at time T5. The switching produces a waveform designated OUT across the two output lines and the peak to peak voltage is twice the supply voltage VDC.

Thus several mechanisms are provided to be able to provide stimulation pulses over a wide range of voltage levels. The first mechanism is the voltage level across the storage capacitor 54, which results from rectifying the pulse width modulated power pulses 46. The width of the power pulses and thus the voltage supplied by the storage capacitor 54 is regulated by the power transmitter 14. That voltage may be controlled between 2.0 and 5.0 volts, for example, and can be applied directly to the electrodes 20 and 21 when stimulation in that voltage range is desired. For stimulation at a higher level, between 4.0 and 10.0 volts for example, bipolar intensification can be employed. Even higher voltage levels can be provided using the flying capacitor converter for voltage intensification which can produce voltages in excess of 10.0 volts depending upon the number of stages.

Determination of the voltage level, shape, and duty cycle of stimulation pulses which are applied to the electrodes 20 and 21 is made by the control circuit 55 in response to physiological characteristics detected by sensor electrodes 57. The stimulation electrodes 20 and 21 also are used for sensing to provide feedback signals for regulating the stimulation. For this purpose, the stimulation electrodes 20 and 21 are connected to inputs of a variable gain instrumentation amplifier 59 with an output that is coupled to an analog input of the control circuit 55. The output signal from the instrumentation amplifier 59 also is applied to an input of a differentiator 53 that has another input which receives a reference signal (REF). The differentiator 53 performs signal transition detection and provides an output to the control circuit 55 that indicates of time events in the sensed physiological data signal.

For example, the differentiator 53 in conjunction with software executed by the control circuit 55 can determine the heart rate and use this information in an algorithm for pacing a patient's heart. The heart rate detection is based on the number of transitions counted over a predefined time interval. If the heart rate goes out of range for a given length of time and the frequency of the transitions remain in the non-fibrillation range, cardiac pacing can be initiated to pace the patient's heart. When the transition frequency indicates fibrillation stimulation for defibrillation can be initiated

When stimulation is occurring, the instrumentation amplifier 119 has low gain (1× or lower) to avoid saturation. When stimulation is inactive (high impedance across stimulation electrodes 20 and 21) as occurs between heart beats, the instrumentation amplifier 119 has a normal gain (100×-200×) to sense physiological characteristics. The gain change is programmably achieved by commands from the control circuit 55 sent to a control port of the instrumentation amplifier 119. The low gain setting allows measurement of the tissue and electrode interface impedance by using the known stimulation pulse duration and amplitude as a known source and the system impedance as a known impedance. From the sensed voltage and the known impedances, the tissue and electrode interface impedance can be determined. This information can also be logged over time to monitor physiological changes that may occur.

For stimulation verification, the control circuit 55 analyzes the sensed parameters to calculate the actual heart rate to determine whether the heart is pacing at the desired rate in response to the stimulation. If the heart is pacing at the desired rate, the control circuit 55 can decrease the stimulation energy in steps until stimulation is no longer effective. The stimulation energy then is increased until the desired rate is achieved. Energy reduction can be accomplished at least in two ways: (1) preferably, the duty cycle is reduced to linearly decrease that amount of energy dissipated in the tissue, or (2) the voltage amplitude is reduced in situations where energy dissipation might vary non-linearly because the tissue/electrode interface is unknown.

The stimulation is controlled by a functionally closed feedback loop. When stimulation commences, the sensed signal waveform can show a physiological response confirming effectiveness of that stimulation pulse. By stepwise increasing the stimulation pulse duration (duty cycle), a threshold can be reached in successive steps. When the threshold is reached, an additional duration can be added to provide a level of insurance that all pacing will occur above the threshold, or it may be sufficient to hold the stimulation pulse duration at the threshold.

After each successful stimulation pulse, a determination is made regarding the difference in duration existing between the last non-effective pulse and the present effective pulse. That difference in duration is added to the present time. The system then senses the effectiveness of subsequent stimulation pulses and remains at the same level for either an unlimited duration or backs off one step in pulse duration. When the effectiveness is maintained again after a preset time window, which could be a number of beats, minutes or hours, the system backs off one decrement at a time. As soon as the effectiveness of the stimulation pulses is lost, the system keeps incrementing the duration until an effective pulse is obtained. In summary, the sensing and stimulation is a closed loop system with two feedback responses: the first response is following an effective pulse and involves gradual reduction of duration after a predetermined number of beats or a predetermined time interval; and the second response is to an ineffective pulse and is immediate with pulse duration adjustment occurring within one beat.

### Supplied Power Control

Another feedback control loop is employed to regulate the electrical power supplied to the implanted medical device 15 from the power transmitter 14. As mentioned previously, the rectifier 50 in the discriminator 49 of the medical device 15 extracts energy from the received first wireless signal 51 to charge the storage capacitor 54. FIG. 7B shows the DC voltage produced by the rectifier 50. The extracted energy charges the storage capacitor 54 that supplies electrical power to components of the implanted medical device 15. The storage capacitor is chosen so that it cannot follow the data stream, and just build up charge. The storage capacitor 54 preferably is a supercapacitor (supercap) that is an electrochemical double layer capacitor (EDLC) hybrid between a conventional capacitor and a battery, and accordingly can be used in place of a battery to extend the life span and power capability of the storage device. However, a battery could be employed as the storage device in place of capacitor 54. In either case, the circuitry of the medical device 15 will receive is power for an extended period even if the power transmitter 14 is not worn by the patient for short periods.

The DC voltage produced by rectifier 50 is regulated. For this function, the DC voltage is applied to a feedback transmitter 63 comprising a voltage detector 62 and a voltage controlled, first radio frequency oscillator 64. The voltage detector 62 senses and compares the DC voltage to a nominal voltage level desired for powering the medical device 15. The result of that comparison is a control voltage which indicates the relationship of the actual DC voltage derived from the received first wireless signal 51 to the nominal voltage level. The control voltage is fed to the input of the voltage controlled, first radio frequency oscillator 64 which produces an output signal at a radio frequency that varies as a function of the control voltage. For example, the first radio frequency oscillator 64 has a center, or second frequency F2 from which the actual output frequency varies in proportion to the polarity and magnitude of the control signal and thus deviation of the actual DC voltage from the nominal voltage level. For example, the first radio frequency oscillator 64 has a first frequency of 100 MHz and varies 100 kHz per volt of the control voltage deviation with the polarity of the control voltage determining whether the oscillator frequency decreases or increases from the second frequency F2. For this exemplary oscillator, if the nominal voltage level is five volts and the output of the rectifier 50 is four volts, or one volt less than nominal, the output of the voltage controlled, first radio frequency oscillator 64 is 99.900 MHz (100 MHz-100 kHz). That output is applied through a first RF amplifier 66 to a first transmit antenna 67 of the implanted medical device 15, which thereby emits a second wireless signal 68.

To control the energy of the first wireless signal 51, the power transmitter 14 contains a second receive antenna 74 that picks up the second wireless signal 68 from the implanted medical device 15. Because the second wireless signal 68 indicates the level of energy received by medical device 15, this enables power transmitter 14 to determine whether medical device requires more or less energy to adequately powered. The second wireless signal 68 is sent from the second receive antenna 74 to a feedback controller 75 which comprises a frequency shift detector 76 and a proportional-integral (PI) controller 80. The second wireless signal 68 is applied to the frequency shift detector 76 which also receives a reference signal at the second frequency F2 from a second radio frequency oscillator 78. The frequency shift detector 76 which acts as a receiver by comparing the frequency of the received second wireless signal 68 to the second frequency F2 and produces a deviation signal ΔF indicating a direction and an amount, if any, that the frequency of the second wireless signal is shifted from the second frequency F2. As described previously, the voltage controlled, first radio frequency oscillator 64, in the medical device 15, shifts the frequency of the second wireless signal 68 by an amount that indicates the voltage from rectifier 50 and thus the level of energy derived from the first wireless signal 51 for powering the implanted medical device 5.

The deviation signal ΔF is applied to the input of the proportional-integral controller 80 which applies a transfer function given by the expression GAIN/(1+sᵢ·τ), where the GAIN is a time independent constant gain factor of the feedback loop, τ is a time coefficient in the LaPlace domain and Sᵢ is the LaPlace term containing the external frequency applied to the system The output of the proportional-integral controller 80 is an error signal indicating an amount that the voltage (VDC) derived by the implanted medical device 15 from the first wireless signal 51 deviates from the nominal voltage level. That error signal corresponds to an arithmetic difference between a setpoint frequency and the product of a time independent constant gain factor, and the time integral of the deviation signal. Other types of feedback controllers may be employed.

The error signal from the feedback controller 75 is sent to the control input of a pulse width modulator (PWM) 82 within a power transmitter 73. The pulse width modulator 82 produces an output signal comprising pulses having a duty cycle that varies from 0% to 100% as dictated by the inputted error signal. The output signal from the pulse width modulator 82 is applied to an input of a second mixer 85 that also received the first radio frequency signal at the first frequency F1 (e.g.<50 MHz) from a second radio frequency oscillator 78. The greater the duty cycle the more energy is transferred to the medical device 15. For example, a 100% duty cycle means that the first radio frequency signal is transmitted continuously and for a 25% duty cycle, the first radio frequency signal is transmitted 25% of each pulse cycle period, and off for 75% of the pulse cycle. The length of each cycle period is a function of the amount of permissible ripple in the first wireless signal 51. For example, a 100 μs cycle period is adequate for a first frequency F1 of 10 MHz. In this case, within one 100 μs cycle and 25% duty cycle, the on-time would be 25 μs containing 250 cycles of the 10 MHz signal. The output from the pulse width modulator 82 is fed to a second data modulator 84 which modulates the signal with configuration commands and data for the medical device 15, as will be described.

The resultant signal is amplified by a radio frequency power amplifier 86 an applied to the transmit antenna 88 which may be of the type described in U.S. Pat. No. 6,917,833. The antennas 74 and 88 in the power transmitter 14 are contained within a patch or arm band 22, shown in FIG. 1, worm on the patient's upper arm 23. The antennas are connected to a module 79 that contains the remainder of the electronic circuitry for the power transmitter 14. The power transmitter 14 is powered by a battery 70, which depending upon its size, may be contained in a separate housing worn elsewhere by the patient.

### Medical Device Configuration

In addition to sending electrical energy to the implanted medical device 15, the power transmitter 14 transmits operational commands and data that configure the functionality of that device or amend the software program that is executed. The implanted medical device 15 also sends operational data to the power transmitter. A data input device, such as a personal computer 90, enables a physician or other medical personnel to specify operating parameters for the implanted medical device 15. Such operating parameters may define the duration of each stimulation pulse, an interval between atrial and ventricular pacing, and thresholds for initiating pacing. When the medical device is intended to stimulate other regions of the patient's body, the operating parameters define the characteristics of that stimulation. The data defining those operating parameters are transferred to the power transmitter 14 via a connector 92 for the input of a serial data interface 94. The data received by the serial data interface 94 can be applied to a microcomputer based control circuit 95 or stored directly in a memory 96.

When new operating parameters are received, the control circuit 95 initiates a transfer of those parameters from the memory 96 to the data input of the second data modulator 84, which also receives the output signal from the pulse width modulator 82. The duty cycle of that output signal varies depending upon the desired magnitude of the electrical energy to be sent to the implanted medical device 15. The second data modulator 84 modulates the output signal to encode the operating commands and data. The resultant composite signal is then transmitted via the RF power amplifier 86 and the transmit antenna 88 to the implanted medical device 15 as the first wireless signal 51.

When the first wireless signal 51 is received by the medical device 15, the data detector 56 recovers operating commands and data as described previously. The control circuit stores the operating parameters for use in controlling the medical device.

Furthermore, the control circuit may include additional sensor electrodes 57 for physiological characteristics of the patient 11, such as heart rate or pressure within the blood vessel in which the medical device 15 is implanted. The sensed data is transmitted from the implanted medical device 15 to the power transmitter 14 via the second wireless signal 68. Specifically, the control circuit 55 sends the physiological data to the first data modulator 65 which produces a signal that is applied to the first RF amplifier 66 to amplitude modulate the signal from the voltage controlled, first radio frequency oscillator 64 with that data.

Data specifying operational conditions of the implanted medical device 15 also can be transmitted via the second wireless signal 68. For example, if the implanted medical device 15 fails to receive the first wireless signal 51 for a predefined period of time. The control circuit 55 generates alarm data which it transmitted via the second wireless signal 68 to alert a data receiver outside the patient of a malfunction of the cardiac pacing system 10. When the power transmitter 14 receives the second wireless signal 68, the data receiver 99 extracts data which then is transferred to the control circuit 95 for storage in memory 96.

FIG. 8A shows the received second wireless signal 68 at the input of the data receiver 99. The square waves in that signal occur at the second radio frequency which was frequency modulated to indicate the DC voltage level in the implanted medical device 15. The physiological data sensed by the medical device 15 also is carried by the second wireless signal 68 digitally as a series of binary bits. Specifically each “1” bit is encoded by a pulse 48 of the first radio frequency signal for a fixed duration bit interval, and each “0” bit is encoded an absence of the radio frequency signal for the bit interval. In other words, the second wireless signal 68 is 100% amplitude modulated for a “1” bit and has zero modulation to represent a binary “0”. The space required for 100/0% AM does not require any additional components as all that is required connector disconnect the output of the first radio frequency oscillator 64 to the first transmit antenna 67.

Other modes of modulation can be used to encode the physiological data. For example, frequency shift keyed (FSK) modulator would require a tone to be mixed into the oscillator (i.e. 2 kHz and 4 kHz). This means that for each “0” and “1”, the control circuit would have to self generate these waveforms. This is, however, power intensive since it requires a continuous control circuit operation. Other ways of modulation may include phase modulation. A version of this may be implemented by “bumping” the oscillator by +ΔF and −ΔF. In this embodiment, one may use the inertia of the receiving tracking phased locked loop (PLL) to create a “steady state” on the patch and add/subtract ΔF representing “0” and “1” at a much faster rate.

Upon interpreting the data as indicating an alarm condition, control circuit 95 activates an alarm, such as by producing an audio signal via a speaker 98 or activate light emitters to produce a visual indication of the alarm. An alarm indication also can be sent via the serial data interface 94 to an external device, such as personal computer 90 for further analysis and storage. In other situations, a wireless communication apparatus, such as a cellular telephone, may be integrated into the power transmitter 14 to transmit an alarm signal to a central monitoring facility.

The foregoing description was primarily directed to preferred embodiments of the invention. Even though some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. An apparatus for artificially stimulating internal tissue of an animal, said apparatus comprising:
an intravascular medical device for implantation in blood vasculature of the animal and comprising a power supply, first and second stimulation electrodes for contacting the tissue, a control circuit which controls operation of the intravascular medical device, a stimulation signal generator connected to the control circuit for producing an electrical stimulation pulse and a voltage intensifier which receives and increases the voltage of the electrical stimulation pulse to produce an output pulse that is applied to the first and second stimulation electrodes.

2. The apparatus as recited in claim 1 wherein the voltage intensifier comprises a flying capacitor type voltage doubler.

3. The apparatus as recited in claim 1 wherein the voltage intensifier comprises a plurality of flying capacitor type voltage doubler stages connected in series.

4. The apparatus as recited in claim 1 wherein the voltage intensifier comprises a flying capacitor type voltage inverter.

5. The apparatus as recited in claim 1 wherein the voltage intensifier increases the voltage of the electrical stimulation pulse using bipolar mode operation.

6. The apparatus as recited in claim 1 further comprising a sensor circuit connected to the first and second stimulation electrodes and having an output at which a feedback signal is produced indicating effects from the electrical stimulation pulse.

7. The apparatus as recited in claim 6 wherein the control circuit responds to the feedback signal by altering the electrical stimulation pulse.

8. The apparatus as recited in claim 1 further comprising an instrumentation amplifier having inputs connected to the first and second stimulation electrodes and having an output connected to the control circuit to provide a feedback signal indicating effects from the electrical stimulation pulse.

9. The apparatus as recited in claim 8 wherein the instrumentation amplifier has a variable gain that is altered by the control circuit.

10. The apparatus as recited in claim 8 wherein the instrumentation amplifier has a lower gain while a stimulation pulse is being applied to the first and second stimulation electrodes than at other times.

11. The apparatus as recited in claim 8 wherein the control circuit varies a characteristic of a subsequent stimulation pulse in response to the feedback signal from the instrumentation amplifier.

12. The apparatus as recited in claim 8 wherein the control circuit analyzes the feedback signal acquired during stimulation to determine efficacy of the stimulation over time.

13. The apparatus as recited in claim 1 further comprising an extravascular power supply comprising a source of electrical power, and a power transmitter connected to the source and emitting a first wireless signal; and
wherein the intravascular medical device further comprises a first receiver for the first wireless signal, and a power circuit that extracts energy from the first wireless signal furnishes that energy to the power supply for powering the medical device.

14. The apparatus as recited in claim 13 wherein:
intravascular medical device further comprises a feedback transmitter that transmits a second wireless signal which indicates an amount of energy extracted from the first wireless signal; and
extravascular power supply further comprises a second receiver for the second wireless signal, wherein the power transmitter alters the first wireless signal in response to the second wireless signal.

15. The apparatus as recited in claim 1 wherein the stimulation pulse is configured to stimulate the internal tissue for cardiac stimulation, GERD treatment, endotracheal stimulation, pelvic floor stimulation, treatment of obstructive airway disorder and apnea, molecular therapy delivery, Parkinson's disease treatment, chronic constipation treatment, obsessive compulsive disorder treatment, or bone healing.

16. The apparatus as recited in claim 1 wherein the internal tissue for stimulation is selected from a list consisting of brain tissue, cardiac tissue, spine tissue, gastro/intestinal tract tissue, pancreas tissue, sacral nerve, muscle tissue, pelvic tissue and bone tissue.

17-29. (canceled)
